(19) United States
(12) Patent Application Publication
(10) Pub. No.: US 2003/0036100 A1
(43) Pub. Date: Feb. 20, 2003

(54) Simultaneous determination of phenotype and genotype

(75) Inventors: Nicholas Maxwell Fisk, London (GB); Phillip Robert Bennett, London (GB); Mahesh Choolani, London (GB)

Correspondence Address: DARBY & DARBY P.C., P. O. BOX 5257, NEW YORK, NY, 10150-5257, US

(73) Assignee: Imperial College Innovations Ltd.

(21) Appl. No.: 10/120,765

(22) Filed: Apr. 10, 2002

Related U.S. Application Data
(60) Provisional application No. 60/282,795, filed on Apr. 10, 2001.

(51) Int. Cl. G01N 33/567; G01N 33/53; C07K 16/46

(52) U.S. Cl. 435/7.21; 435/7.5; 530/391.1

(57) ABSTRACT

The invention provides antibody which can bind to an antigen present in a heme-containing cell and to which a fluorophore with an emission wavelength of between 420 nm and 500 nm is bound. The antibody of the invention can bind to an antigen of interest in a heme-containing cell and, due to the fluorophore, the binding interaction can be visualized. This facilitates the use of fluorescent labels for immunophenotyping of red blood cells and, in particular, fetal red blood cells within a blood sample from a pregnant female. It also facilitates simultaneous use of immunophenotyping and fluorescence-based nucleic acid analytical techniques such as FISH. Genotype and phenotype can be detected at the same time.

This patent application claims priority under 35 U.S.C. §119(e) to U.S. provisional patent application Serial No. 60/282,795, filed Apr. 10, 2001, which is incorporated herein by reference in its entirety.

### TECHNICAL FIELD

This invention is in the field of in situ analysis of cells using fluorophores. In particular, it relates to the analysis of fetal red blood cells.

### BACKGROUND ART

Fetal nucleated red blood cells (NRBCs) can escape into maternal circulation [e.g. 1] and these cells are useful for non-invasive prenatal genetic testing. Two fundamental steps are involved in this: identification of fetal cells among background maternal cells, followed by genetic testing of the identified fetal cells.

The first step, in which fetal cells are identified, typically uses immunophenotyping. This is a well-known technique [e.g. refs. 2 to 5], also referred to as immunocytochemistry, involving the use of labeled antibodies which are specific to cellular markers. Binding of the antibody to the markers is typically assessed by microscopy, or may be used as the basis of cell sorting (e.g. in FACS). To differentiate fetal cells from maternal cells, immunophenotyping using labeled antibody against the γ-chain of fetal hemoglobin (HbF) is commonly used [e.g. 6, 7, 8].

The second step, in which the fetal cells are genetically tested, often involves fluorescence in situ hybridization (FISH). This is another well-known technique, involving hybridizing a labeled nucleic acid probe to the chromosomes of a cell [3, 9, 10].

Rare event detection poses several problems, the greatest being the loss of already-scarce fetal cells when these steps are carried out separately. Furthermore, the separation of identification and diagnostic steps has the potential for error, especially if there is a need to switch microscopes [11] or to relocate the cell of interest [12]. It would therefore be desirable to perform these two steps at the same time (i.e. to assess the phenotype and genotype of cells in a maternal blood sample simultaneously) in order to minimize fetal cell loss and the scope for error. Previous strategies for combining the steps [e.g. 8, 11, 13 to 19] have proved unsatisfactory. One problem is due to the dyes typically used in immunophenotyping—Fast Red™ dissolves in the organic solvents used in FISH, for instance, and Vector Blue Substrate™ decreases hybridization efficiency and thus FISH sensitivity. A further problem is that acetic acid, commonly used to fix cells, results in altered cell morphology. Another problem is that combining the techniques necessitates either the recording of spatial orientation of fetal cells and relocation during subsequent analysis, or cumbersome switching between light and fluorescence microscopes [11]. To obviate this, it has been proposed [6] to use fluorophores to label fetal antigens rather than the usual light microscopy dyes.

The use of fluorophores in fetal erythroid cells is hampered, however, by heme autofluorescence and the overlap of colour signals with those used for FISH [6]. This problem has been recognized for several years and two solutions have previously been proposed. A first solution simply subtracts the background autofluorescence mathematically [20] and, whilst this is effective, it is not sufficiently sensitive, accurate or reliable for use in situations which require a high degree of certainty (e.g. in the diagnosis of life-threatening diseases). A second solution has been to quench the autofluorescence [21], but this is also insufficiently sensitive for critical applications.

It is an object of the invention to facilitate the use of fluorescent labels for accurate immunophenotyping of red blood cells. It is a further object to facilitate the simultaneous visualisation of protein markers and nuclear FISH signals in red blood cells.

### SUMMARY OF THE INVENTION

The invention is based around the use of a fluorophore with an emission wavelength of between 420 nm and 500 nm as a label for immunophenotyping.

The invention provides antibody which can bind to an antigen present in a heme-containing cell and to which a fluorophore with an emission wavelength of between 420 nm and 500 nm is bound. The antibody of the invention can bind to an antigen of interest in a heme-containing cell and, due to the fluorophore, the binding interaction can be visualized.

The invention also provides a method for detecting cells which express an antigen of interest, comprising the steps of: (a) contacting a sample with antibody of the invention; and (b) detecting the binding of fluorophore to cells within the sample. The method is preferably for identifying fetal cells from within a mixture of fetal and maternal cells (e.g. within a maternal blood sample), and may comprise the further step of genetically testing a detected cell.

The invention also provides a method for identifying a fetal heme-containing cell in a blood sample taken from a pregnant female, comprising the steps of: (a) contacting the blood sample with antibody of the invention, wherein said antibody is specific for fetal cells; (b) identifying the cells in the sample to which fluorophore binds. The invention also provides a method for simultaneously detecting the phenotype and genotype of a cell, comprising the steps of: (a) contacting the cell with antibody of the invention; (b) contacting the cell with a nucleic acid probe labeled with a fluorophore; and (c) detecting the fluorophores.

The invention also provides a method for genetically testing fetal cells within a blood sample from a pregnant female, comprising the steps of: (a) contacting the sample with antibody of the invention; (b) contacting the sample with a nucleic acid probe; and (c) detecting the antibody and the probe. The antibody distinguishes fetal and maternal cells by immunophenotype; the nucleic acid probe analyses the genotype of cells within the sample. Their simultaneous use thus allows the genotype of fetal cells in the sample to be assessed.

### BRIEF DESCRIPTION OF DRAWINGS

FIG. 1 shows the relative fluorescence intensity of stained (A, B, C) and unstained (D1, D2, D3) fetal cells through Green (A, D1), Red (B, D2) and Blue (C, D3) channels. Mean RFIC values and 95% data intervals (±1.96 SD) are shown.

FIGS. 2A-2B shows regression analysis of (A) the fall in the percentage of fetal erythroblasts which express ε-globin and (B) the fall in fetal primitive erythroblasts as a proportion of the nucleated cell count, both with respect to gestational week during the first trimester.

FIGS. 3A-3H (color photograph) shows the simultaneous visualization of (i) ε-globin as an intracytoplasmic fetal cell identifier and (ii) chromosomal FISH.

FIG. 4 (color photograph) is similar, but the fetal cell that is visible was purified from a blood sample taken from a pregnant female.

FIGS. 5A-5C (color photograph) shows a male and a female fetal erythroblast purified from blood samples from pregnant females and analyzed by simultaneous immunophenotyping and chromosomal FISH. Red and green signals are from X- and Y-chromosome probes, respectively.

### DETAILED DESCRIPTION OF THE INVENTION

Fluorophores for use with the Invention

The invention uses fluorophores with an emission wavelength of between 420 nm and 500 nm as labels for immunophenotyping [22]. Preferred ranges within the emission wavelength lies are 420-490 nm, 420-480 nm, 420-470 nm, 420-460 nm, 420-450 nm, 430-500 nm, 430-490 nm, 430-480 nm, 430-470 nm, 430-460 nm, 430-450 nm, 440-500 nm, 440-490 nm, 440-480 nm, 440-470 nm, 440-460 nm, 440-450 nm, 450-500 nm, 450-490 nm, 450-480 nm, 450-470 nm, and 450-460 nm. The emission wavelength is more preferably between 440 nm and 450 nm, and most preferably around 445 nm.

Preferred fluorophores for use with the invention are 7-aminocoumarin derivatives. A particularly preferred fluorophore is 7-amino-4-methylcoumarin-3-acetic acid (AMCA). Other suitable fluorophores include 4-methylumbellierone, Calcofluor™ White, Cascade Blue, and BFP, as well as AMCA derivatives such as Alexa Fluor 350 (sulphonated AMCA derivative), AMCA-X (6-((7-amino-4-methylcoumarin-3-acetyl)amino)hexanoic acid) and AMCA-S (7-amino-3-((((succininimidyl) oxy)carbonyl)methyl)-4-methylcoumarin-6-sulfonic acid). Hydroxycoumarins may also be used, particularly fluorinated 7-hydroxycoumarins such as Marina Blue (6,8-difluoro-7-hydroxy-4-methylcoumarin) and Pacific Blue (3-carboxy-6,8-difluoro-7-hydroxycoumarin). Fluorophores which intercalate DNA (e.g. Hoechst 33342, DAPI, POP etc.) should only be used if conjugated, modified or manipulated such that they cannot interact with nucleic acid. Preferably, therefore, the fluorophore does not bind to DNA.

The use of a fluorophore with an emission wavelength of between 420 nm and 500 nm is advantageous for several reasons. First, red and green fluorophores have been found by the inventors to be unsuitable for labeling intracytoplasmic globins because, in a significant proportion of cases, stained and unstained cells cannot be distinguished due to autofluorescence of the cells. Second, red and green labels are commonly used to label FISH probes [6], so the fluorophore need not interfere with FISH. Third, whilst blue is the color most commonly reserved for nuclear counterstain in chromosomal FISH, the antibody of the invention can advantageously avoid the need for such counterstain on intact cells because it can act as a surrogate counterstain itself.

The excitation wavelength of the fluorophore is not critical.

Labeled Antibody for Immunophenotyping

The invention provides antibody which can bind to an antigen present in a heme-containing cell and to which a fluorophore with an emission wavelength of between 420 nm and 500 nm is bound.

The fluorophore may be bound to an antibody directly. This involves a covalent linkage between the fluorophore and the antibody which binds to the antigen. This can be achieved by conjugation via covalent linkage to an amino acid side chain, for instance. Methods for attaching fluorophores to antibody are well known [e.g. 22, 23, 24 etc.] and may involve, for instance, the use of a succinimidyl ester of the fluorophore.

In current immunophenotyping methods, however, it is more usual for the fluorophore to be bound to the antibody indirectly i.e. the antibody which binds to the antigen (first antibody) is different from the molecule to which the fluorophore is covalently attached. Generally, indirect arrangements of this type have the fluorophore attached to a second antibody which binds to the first antibody (e.g. the first antibody is a murine antibody, and the second antibody is anti-mouse). The fluorophore may in turn be directly or indirectly bound to the second antibody. A typical arrangement has a first antibody which binds to the antigen, a second antibody which binds to the first antibody and to which a ligand is bound (e.g. biotin), and an anti-ligand (e.g. avidin or streptavidin) to which the fluorophore is covalently bound.

Thus the invention provides an antibody which can bind to an antigen present in a heme-containing cell and to which a fluorophore with an emission wavelength of between 420 nm and 500 nm is covalently bound.

The invention also provides a kit comprising (a) a first antibody which can bind to an antigen present in a heme-containing cell; (b) a second antibody which can bind to the first antibody and to which a fluorophore with an emission wavelength of between 420 nm and 500 nm is covalently bound.

The invention also provides a kit comprising (a) a first antibody which can bind to an antigen present in a heme-containing cell; (b) a second antibody which can bind to the first antibody and to which a ligand is bound; and (c) an anti-ligand to which a fluorophore with an emission wavelength of between 420 nm and 500 nm is covalently bound. The ligand and anti-ligand are preferably biotin and avidin/streptavidin.

It will be appreciated that the term ‘antibody’ may include polyclonal and monoclonal antibodies, antibody fragments (eg. F(ab)₂, Fab′, Fv, etc.), single chain antibodies (sFv etc.), recombinant antibodies, engineered antibodies, chimeric antibodies, humanized antibodies etc., provided that the relevant antigen reactivity is retained. Monoclonal antibodies are preferred for binding to the antigen present in a heme-containing cell. Many suitable antibodies are commercially available.

Heme-Containing Cells

The heme-containing cell will generally be a human cell. It is preferably a fetal cell, and most preferably a fetal erythroid cell. Typical erythroid cells are erythrocytes and erythroblasts.

The cell is preferably nucleated, and is more preferably mononuclear.

Fetal-Specific Antigens

The antigen present in the heme-containing cell is preferably hemoglobin or a subunit thereof (i.e. the α, β, γ, δ, ε or ζ chain subunits). This is a commonly used cytoplasmic antigen for immunophenotyping.

The antibody of the invention preferably binds to fetal cells with higher affinity than to maternal cells. Accordingly, the antigen is preferably a hemoglobin subunit which is found in fetal or embryonic hemoglobin but not in adult hemoglobin (i.e. the γ, ε or ζ chain subunits).

Whilst the γ chain is commonly used [e.g. 6, 7, 8], this chain is also maternally expressed during pregnancy [25] and in β-thalassemia [26, 27]. Preferably, therefore, the antigen is the 4 or, more preferably, the ε chain subunit of hemoglobin [28]. The ε chain subunit (‘ε-globin’) has been suggested as the ideal marker for fetal erythroblast identification [29 to 32] and this is a suitable antigen for detecting fetal cells in the first 14 weeks of pregnancy, being expressed exclusively in primitive NRBCs.

Immunophenotyping Methods

The invention provides a method for identifying a cell which expresses an antigen of interest, comprising the steps of: (a) contacting a sample containing a cell or cells with antibody of the invention; and (b) identifying a cell or cells within the sample to which fluorophore is bound.

Where the fluorophore is bound indirectly to the antibody which binds to the antigen of interest, step (a) may involve contacting the cell in turn with (i) a first antibody which binds to the antigen of interest, (ii) a second antibody which binds to the first antibody and to which the fluorophore is covalently bound. In an alternative format, step (a) may involve contacting the cell in turn with (i) a first antibody which binds to the antigen of interest, (ii) a second antibody which binds to the first antibody and to which a ligand is bound, and (iii) an anti-ligand to which the fluorophore is covalently bound.

This method may be for confirming whether a particular cell or cell population expresses the antigen of interest, or it may be for identifying particular cells within a mixed population.

The method is preferably used to identify fetal cells from within a mixture of fetal and maternal cells (e.g. from within a maternal blood sample). The antigen of interest in this case will be a fetal-specific antigen.

The method is preferably performed on a solid substrate e.g. on a microscope slide etc.

The method of the invention may comprise the further step of performing genetic tests on a cell identified as expressing an antigen of interest. Thus the invention provides a method for genetically testing a cell, comprising the steps of: (a) contacting a sample containing a cell or cells with antibody of the invention; (b) selecting a cell or cells within the sample to which fluorophore is bound; and (c) performing genetic tests on said cell or cells.

Identifying Fetal Cells in Maternal Blood Samples

The invention also provides a method for identifying a fetal cell in a blood sample taken from a pregnant female, comprising the steps of: (a) contacting the blood sample with antibody of the invention, wherein said antibody can bind to an antigen which is expressed by fetal cells within the sample but not by maternal cells within the sample; (b) identifying a cell or cells in the sample to which fluorophore is bound. These cells may be subjected to genetic testing.

Any blood sample from a pregnant female (preferably a human) can be used, but it is preferred that the sample should be taken during the first trimester (weeks 0-13) of pregnancy. More preferably the blood sample is taken during weeks 5-13, and most preferable weeks 7-13.

Typical samples will be venous blood, with a volume between 1 and 50 ml (e.g. 20-30 ml).

The blood sample may be enriched for nucleated fetal erythroid cells before it is contacted with antibody of the invention [e.g. 33].

Simultaneous Immunophenotyping and Genotyping

The invention also provides a method for simultaneously detecting the phenotype and genotype of a cell, comprising the steps of: (a) contacting a cell or cells with antibody of the invention, which is labeled with a first fluorophore; (b) contacting the cell or cells with a nucleic acid probe, which is labeled with a second fluorophore; and (c) detecting a cell or cells in the sample to which said first and second fluorophores are bound. Steps (a) and (b) can be carried out in either order, but step (a) preferably precedes step (b) for better results.

Detection is ‘simultaneous’ in that the various signals from the fluorophores are emitted at the same time. The signals need not physically be detected by a user at precisely the same time, but the need for using separate techniques for assessing genotype and immunophenotype is avoided, so the signals can be detected in the same experiment using the same instrument. Detection can thus be formed in parallel, rather than in series.

Step (b) may involve contacting the cell with a plurality of nucleic acid probes, each of which may be labeled with a different fluorophore. Use of fluorescent probes in parallel is well known [e.g. 34, 35].

It is preferred that the first fluorophore gives a signal of a different color from the second and any further fluorophores, as the phenotype and genotype signals can be more easily differentiated. The emission wavelength(s) of the second and any further fluorophores are preferably more than 10 nm different (e.g. longer than) the emission wavelength of the first fluorophore. The difference is preferably more than 20 nm (e.g. >30 nm, >40 nm, >50 nm, >60 nm, >70 nm, >80 nm, >90 nm, >100 nm, >110 nm, >120 nm, >130 nm, >140 nm, >150 nm, >160 nm, >170 nm, >180 nm, >190 nm, >200 nm, >210 nm, >220 nm, >230 nm, >240 nm, or >250 nm). The second and any further fluorophores preferably do not emit signals in the blue region of the spectrum, with the red, orange, yellow and green regions being preferred. Preferred fluorophores for use with probe(s) are SpectrumOrange™ and SpectrumGreen™.

Fluorophores may be bound to the probe(s) directly, involving a covalent linkage between the fluorophore and the nucleic acid. This can be achieved by conjugation via covalent linkage to nucleotides, for instance. As an alternative, they may be bound to the probe indirectly e.g. in branched DNA assays [36], or via a ligand/anti-ligand interaction (e.g. biotin/streptavidin).

The probes may comprise RNA, DNA, PNA, or mixtures thereof.

The nucleic acid probes are suitable for in situ detection of genetic material of interest. Preferred probes are specific for the X or Y chromosomes or for chromosomes 13, 18 or 21.

Genetic Testing of Fetal Cells in a Maternal Blood Sample

The invention also provides a method for genetically testing fetal cells within a blood sample from a pregnant female, comprising the steps of: (a) contacting the sample with antibody of the invention, wherein said antibody is labeled with a first fluorophore and binds to an antigen which is expressed by fetal cells within the sample but not by maternal cells within the sample; (b) contacting the sample with a nucleic acid probe, wherein the probe is labeled with a second fluorophore and is specific for a genetic disorder; and (c) detecting a cell or cells in the sample to which said first and second fluorophores are bound.

The antibody of the invention used in step (a) distinguishes fetal and maternal cells by immunophenotype; the nucleic acid probe used in step (b) analyses the genotype of cells within the sample. The simultaneous use of antibody and probe thus allows the genotype of fetal cells in the sample to be assessed.

The overall procedure can be performed in less than 8 hours, has a very high hybridization efficiency, 100% specificity and a sensitivity of 1 in 10⁵ nucleated cells and 1 in 10⁶ RBCs.

Genetic Testing Techniques

When cells have been identified using antibody of the invention, they are preferably subsequently tested for genotype and/or genetic disorders. The sex of the fetus can also conveniently be determined.

These tests are preferably performed on the cell in situ, but the cell can be removed for further analysis. Preferred in situ methods which may be used are in situ RNA hybridization, in situ PCR [37] and FISH, although any technique based on nucleic acid hybridization may be used. FISH using single or multiple probes and single or multiple fluorophores is preferred.

Any disorder which is known to have a genetic basis may be tested, including disorders with a chromosomal abnormality [e.g. Down's Syndrome (trisomy 21), Turner's Syndrome (XO chromosomes), Klinefelter's Syndrome (XXY), Edward's Syndrome (trisomy 18), Patau Syndrome (trisomy 13)] and single-gene disorders [e.g. cystic fibrosis, alpha and beta thalassemia, hemophilia, muscular dystrophy, myotonic dystrophy, sickle cell disease, Huntington disease etc.].

The test may be at a chromosomal level, or may require more detailed genetic analysis (e.g. nucleic acid separation, RFLP detection, PCR, or sequencing). Preferred methods use fluorescent labels, with FISH being particularly preferred.

Chromosomal FISH is the preferred method [38], including its many variants e.g. ‘m-FISH’ [39], spectral karyotyping (‘SKY’) [40, 41], ‘poly-FISH’ [42], ‘COBRA’ [43], ‘interphase FISH’ [8], ‘Rx-FISH’, ‘chromosome banding’ [35, 44], SNP FISH [45] etc. [46]. Briefly, FISH involves the following steps: (a) pre-hybridization of the cell to increase accessibility of target nucleic acid e.g. denaturation by heat or alkali; (b) an optional step to reduce non-specific binding e.g. by blocking repetitive sequences; (c) hybridization to one or more FISH probes; (d) post-hybridization washing and/or nuclease treatment to remove free probes; and (e) detection of the hybridized FISH probes.

Fluorescence Microscopy

Fluorophores are typically detected using fluorescence microscopy. Preferably, reflected light fluorescence microscopy (epifluorescence) is used.

Suitable filters for visualizing the fluorophore attached to antibody of the invention include Aqua and Blue filters. Conveniently, autofluorescence of heme can be viewed through Aqua filters, allowing AMCA-stained and unstained cells to be distinguished.

Confocal scanning laser microscopy may also be used.

Fixation and Permeabilization

Prior to contacting cells with antibody of the invention (and, optionally, afterwards as well), they are preferably fixed. Various fixatives are known to the skilled person (e.g. formaldehyde, formalin, paraformaldehyde, glutaraldehyde, Bouin's fixative, Camoy's fixative etc.). It is advantageous, however, that the fixative should be essentially free from acetic acid. A preferred fixative is a mixture of methanol and acetone, with the ratio of methanol:acetone typically in the range 3:1 to 1:5 (v/v), preferably around 1:1 (v/v).

Prior to contacting cells with antibody, they are also preferably permeabilized [e.g. 47], particularly where the antigen of interest is cytoplasmic. Various permeabilizing agents are known to the skilled person (e.g. methanol/acetone mixtures, detergents such as Tween 20, Triton X, NP-40, acetone etc.). It is preferred, however, to permeabilize the cells using a very dilute amount of acetic acid. A preferred permeabilizing agent is 0.25% (v/v) glacial acetic acid in methanol.

The avoidance of acetic acid during fixation combined with its very dilute use during permeabilization advantageously allows preservation of the fluorophore label whilst facilitating the unraveling of nuclear histones to facilitate subsequent genetic analysis.

The invention provides a method for permeabilizing cells, wherein the permeabilizing agent consists of a dilute solution of glacial acetic acid. The concentration of glacial acetic acid is less than 2% (v/v), preferably less than 1% (v/v), more preferably less than 0.5% (v/v), and most preferably around 0.25% (v/v). Preferred solvents are C₁, C₂, C₃ or C₄ alcohols, with methanol being particularly preferred. The solution is preferably used for less than 5 minutes.

Cell Sorting Methods

The invention also provides a flow cytometry method [e.g. 48, 49], in which cells are labeled with antibody of the invention. The interaction between heme and the antibody gives rise to a fluorescent signal. The method is preferably fluorescence activated cell sorting (FACS), with heme-containing cells method being sorted on the basis of fluorescence of the antibody.

The method may be used to separate fetal cells from maternal cells e.g. in a blood sample. The immunophenotype and genotype of the separated fetal cells may then be ascertained using the methods of the invention.

### EXAMPLES ^{[see also reference 29]}

Collection of Fetal Blood Samples

Fetal whole blood was obtained by ultrasound-guided transabdominal cardiocentesis before clinically-indicated surgical termination of pregnancy [50]. Blood collection was approved by the institutional ethics committee in compliance with national guidelines regarding the use of fetal tissue for research purposes. All women gave written informed consent. Gestational ages determined by crown-rump length measurement ranged from 7-14 weeks.

Studying Heme Autofluorescence

To determine the limiting effect of heme autofluorescence on choosing a fluorescence label for anti-globin antibody, four groups of 50 fetal erythroblasts from the same sample at 9 week gestation were studied. In 3 groups, cells were stained by fluorescence immunocytochemistry using either fluorescein isothiocyanate (FITC), phycoerythrin (PE) or AMCA for either the ε- or the y-globin chain and the fluorescence intensities of positive cells studied. The fourth group was not stained and the autofluorescence within the cells determined through the Red (Texas Red™), Green (FITC) and Blue (DAPI; diamidino-2-phenyl-indole) channels.

FITC and AMCA were used to label ε-globin (Europa Bioproducts, Cambridge, UK). PE-labeled γ-globin was used because it was available pre-conjugated (Europa Bioproducts).

To compare image intensities, all images were ColourNormalised (256 grey levels; IPLab Software, Digital Scientific, Cambridge, UK) according to set criteria before analysis. Ten randomly selected clusters of 5 neighbouring cells were studied for each of the 4 study groups. Clusters were labeled 1-10 consecutively, upon selection. Within each cell, 10 small areas within the cytoplasm were studied. A mean fluorescence intensity was calculated for each cluster of 5 cells. This number was transformed to a Relative Fluorescence Intensity of Cluster (RFIC) by making it a percentage of the 256 grey levels [20]. Within each filter channel, Green, Red and Blue, the difference between corresponding RFICs was calculated.

The mean difference in the RFICs between stained and unstained erythroblasts for the three stains were as follows:

| | | | Mean RFIC | 95% confidence | Standard |
|---|---|---|---|---|---|
| Stain | | Channel | difference | interval | deviation |
| FITC | A | Green | 24.1 | 16.3-31.9 | 12.4 |
| PE | B | Red | 9.8 | 4.8-14.8 | 8.0 |
| AMCA | C | Blue | 43.0 | 34.2-51.8 | 13.9 |

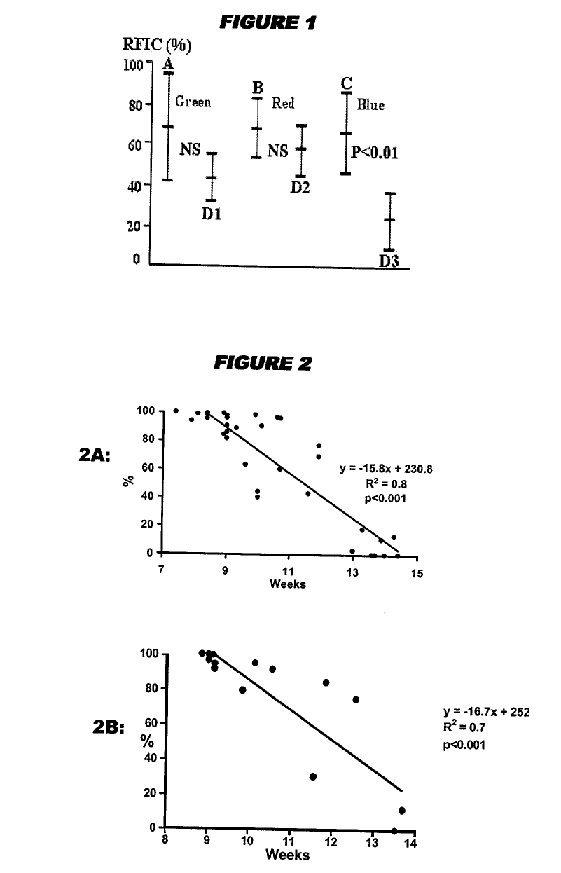

The greatest difference in RFIC was thus achieved using AMCA. As shown in FIG. 1, where D1, D2, D3 represent the autofluorescence of unstained fetal erythroblasts when viewed through the Green, Red and Blue filters respectively, there is overlap between the brighter autofluorescent and weakly stained cells in the Green and Red channels but not in the Blue.

Viewed through the Green channel, 55.9% of unstained cells have a greater (auto)fluorescence intensity than weakly-stained positive cells; this latter group of weakly-stained cells represent 23.8% of all positive cells, and 20.1% of all green cells fall within this zone of ambiguity. Similarly, through the Red channel, 68.7% of unstained cells have a greater (auto)fluorescence intensity than weakly-stained positive cells; this latter group of weakly-stained cells represent 58.2% of all positive cells, and 46.0% of all red cells fall within this zone of ambiguity. In the remaining 79.9% of cases for Green, and 44.1% for Red, stained and autofluorescent cells can be easily distinguished by optimising the fluorescence threshold on the colour-histogram in the image capture software.

In contrast, there is no overlap of fluorescence between stained and unstained cells viewed through the Blue channel, meaning that confusion between positive and negative cells is unlikely. AMCA was therefore the label of choice for the anti-ε globin antibody.

Preparation of Slides

30,000 nucleated cells suspended in 1% BSA in PBS, were cytocentrifuged onto glass slides.

Pure populations of K562 cells and adult bone marrow erythroblasts were used as positive and negative controls, respectively. K562 cells cultured in 0.1 mM hemin express ε-globin [51].

Sensitivity was ascertained in mixtures of male fetal NRBCs within nucleated cells from a never-pregnant female, ratios ranging from 1:10² to 1:10^{5.} Maternal NRBCs enriched from peripheral blood of mothers carrying a male fetus were studied for expression of ε-globin. Specificity was confirmed in mixtures of male fetal erythroblasts with female adult bone marrow erythroblasts.

Cell Fixing and Permeabilization

In order to fix cells onto a glass slide whilst retaining cell morphology and hemoglobin, and not hindering chromosomal FISH hybridization, various fixatives were evaluated.

Cell morphology was graded: “excellent” if there was no perceptible difference between fixed and unfixed fetal erythroblasts, “good” if a clear difference existed but the cells could be easily identified as fetal erythroblasts, “poor” if it was difficult to recognize the cells as fetal erythroblasts, and “absent” if cells were completely disrupted. Chromosomal FISH hybridization efficiencies were broadly categorized: “excellent” for hybridization efficiency above 90%, “good” 65-89%, “fair” 30-64%, and “poor” below 30%. Results were as follows:

| | | Cell | | Hybridization |
|---|---|---|---|---|
| Fixative | | Morphology | Hemoglobin | Efficiency |
| Methanol:glacial acetic acid | 3:1 | Absent | Completely lost | Excellent |
| | 4:1 | Absent | Completely lost | Excellent |
| | 9:1 | Absent | Completely lost | Excellent |
| | 19:1 | Poor | Some Hb intact | Good |
| Paraformaldehyde (2 min) | 4% | Excellent | Completely intact | Poor |
| Formaldehyde (2 min) | 2% | Excellent | Completely intact | Poor |
| | 4% | Excellent | Completely intact | Poor |
| | 5% | Excellent | Completely intact | Poor |
| Methanol:acetone (2 min) | 1:0 | Excellent | Completely intact | Poor |
| | 19:1 | Excellent | Completely intact | Poor |
| | 15:1 | Excellent | Completely intact | Poor |
| | 10:1 | Excellent | Completely intact | Poor |
| | 5:1 | Excellent | Completely intact | Poor |
| | 3:1 | Excellent | Completely intact | Fair |
| | 1:1 | Excellent | Completely intact | Fair |
| | 1:3 | Good | Completely intact | Good |
| | 1:5 | Good | Completely intact | Good |
| | 1:10 | Good | Most Hb intact | Good |
| | 1:15 | Poor | Some Hb intact | Excellent |
| | 1:19 | Poor | Most Hb lost | Excellent |
| | 0:1 | Poor | Most Hb lost | Excellent |

3:1 (v/v) methanol:glacial acetic acid is a standard (Carnoy's) fixative used in FISH that generates excellent hybridization efficiency but severely disrupts the cell membrane and cytoplasm, leaving only the nucleus present and intact on the glass slide. Cross-linkers such as formaldehyde or paraformaldehyde resulted in good cell morphology but poor nuclear hybridization and trapped most of the FISH probe within the cytoplasm. Methanol appeared necessary to retain good cell morphology and cytoplasmic staining. Increasing exposure to acetone improved FISH hybridization signal but damaged cell morphology. Leakage of the hemoglobin onto the intercellular space between the erythroblasts on the glass slide appeared to be directly related to the retention of cell morphology and integrity of the cell membrane.

These initial experiments suggested that 1:1 v/v methanol:acetone may be the optimal fixative. It was postulated that an increased duration of exposure to the acetone within this mixture may permeabilize the cell membrane to improve probe penetration and hybridization efficiency, so the mixture was tested for various fixation times:

| Fixation time | Cell | | Hybridization |
|---|---|---|---|
| (minutes) | Morphology | Hemoglobin | Efficiency |
| 1:00 | Excellent | Excellent | Fair |
| 2:00 | Excellent | Excellent | Fair |
| 3:00 | Excellent | Excellent | Fair |
| 4:00 | Excellent | Excellent | Fair |
| 5:00 | Excellent | Excellent | Fair |
| 6:00 | Good | Most Hb intact | Fair |
| 7:00 | Good | Most Hb intact | Fair |
| 8:00 | Good | Most Hb intact | Good |
| 9:00 | Fair | Most Hb intact | Good |
| 10:00 | Fair | Most Hb intact | Good |
| 15:00 | Fair | Some Hb intact | Excellent |
| 20:00 | Poor | Most Hb lost | Excellent |

Although the best hybridization efficiency was achieved after 15 minutes, cell morphology and hemoglobin content was affected. Incubation for 8 minutes gave the optimal result with some improvement in hybridization.

3% glacial acetic acid is used to differentially lyse erythrocytes when performing a nucleated cell count on a hemocytometer. It was postulated that advantage might be taken of this effect to both lyse unwanted erythrocytes and remove basic proteins within the orthochromatic nucleus of fetal erythroblasts that may be interfering with probe hybridization. The effect of different concentrations of glacial acetic acid and of different exposure times to erythrocytes and fetal erythroblasts was studied in real-time using a hemocytometer. Lysis was graded as follows: “0” no lysis, “1” <50% cells lysed, “2” ≧50% cells lysed, “3” all cells lysed:

| Exposure | | | | | |
|---|---|---|---|---|---|
| time (minutes) | 3% | 2% | 1% | 0.50% | 0.25% |
| Erythroblasts | | | | | |
| 0:30 | 0 | 0 | 0 | 0 | 0 |
| 1:00 | 1 | 0 | 0 | 0 | 0 |
| 2:00 | 1 | 1 | 0 | 0 | 0 |
| 3:00 | 1 | 1 | 1 | 1 | 0 |
| 4:00 | 2 | 2 | 1 | 1 | 0 |
| 5:00 | 2 | 2 | 1 | 1 | 1 |
| 6:00 | 2 | 2 | 2 | 1 | 1 |
| 7:00 | 2 | 2 | 2 | 1 | 1 |
| 8:00 | 2 | 2 | 2 | 2 | 1 |
| 9:00 | 2 | 2 | 2 | 2 | 1 |
| 10:00 | 2 | 2 | 2 | 2 | 1 |
| 15:00 | 3 | 2 | 2 | 2 | 2 |
| 20:00 | | 3 | 2 | 2 | 2 |
| Erythrocytes | | | | | |
| 0:30 | 3 | 3 | 2 | 1 | 1 |
| 1:00 | | | 2 | 1 | 1 |
| 2:00 | | | 2 | 2 | 2 |
| 3:00 | | | 3 | 2 | 2 |
| 4:00 | | | | 2 | 2 |
| 5:00 | | | | 2 | 2 |
| 6:00 | | | | 2 | 2 |
| 7:00 | | | | 2 | 2 |
| 8:00 | | | | 2 | 2 |
| 9:00 | | | | 3 | 2 |
| 10:00 | | | | | 2 |
| 15:00 | | | | | 3 |
| 20:00 | | | | | 3 |

The least effect on fetal erythroblasts was seen using 0.25% for 5 minutes.

Combined Fluorescence Immunocytochemistry and Chromosomal FISH

Immunophenotyping Slides were fixed in 1:1 (v/v) methanol:acetone for 8 minutes at room temperature, permeabilized with 0.25% glacial acetic acid in methanol (v/v) and rinsed in TBST (Tris-buffered saline with Tween-20 (polyoxyethylene sorbitan monolaurate; DAKO Corporation, Carpinteria, Calif.)). Slides were then incubated for 30 minutes with goat serum (Sigma Diagnostics, St. Louis, Miss.) diluted 1:5 in TBST followed by incubation for 60 minutes with anti-ε monoclonal antibody (Europa Bioproducts) diluted 1:100, washing twice after each incubation. Subsequent incubations were with biotinylated goat anti-mouse (Vector Laboratories, Burlingame, Calif.), and with AMCA-conjugated streptavidin (Vector Laboratories), both diluted 1:100 and incubated for 30 minutes. Reagents were diluted in TBST, incubations were in a humidifying chamber at room temperature and washes were in TBST for 3 minutes. Slides were dehydrated through 70%, 90% and 100% ethanol, air dried and prepared for FISH to the sex chromosomes.

Chromosomal FISH The chromosome-specific centromeric repeat probes DXZ1 (labeled with SpectrumOrange™) and DYZ1 (labeled with SpectrumGreen™) were used. 5 μl probe, diluted 1:1 in hybridization buffer, containing 50% formamide and 10% dextran sulphate in 2× SSC at pH 7.0, were added to each cytospin under a coverglass. Target DNA was denatured on an in situ hybridization block at 71° C. for 7 minutes followed by 4 hours hybridization at 37° C. Post-hybridization washes included once in 0.4× SSC at 72° C. for 2 minutes and twice in 2× SSC at room temperature for 2 minutes.

Slides were dehydrated through an ethanol series and mounted in fluorescence antifade medium (Vector Laboratories). The slides were analyzed by epifluorescence microscopy using single band pass filters for SpectrumAqua™ (Aqua) and SpectrumOrange™ (Orange) and a triple band pass filter set for DAPI, FITC and Texas Red™. Images were captured using a cooled charge-coupled device camera and reviewed in Quipps m-FISH software (Vysis, Downer's Grove, Ill.

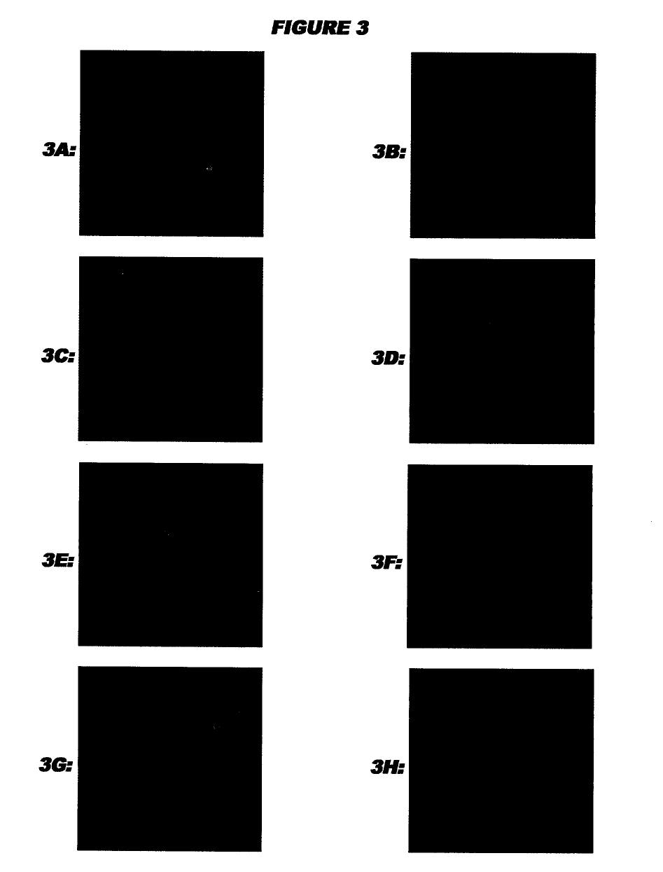

Panel A of FIG. 3 shows ε-positive primitive fetal erythroblast at 10 weeks' gestation stained with AMCA. Cell morphology is well preserved, and this large ε-positive erythroblast is typical for this stage of gestation. No DAPI was used as counterstain, as accumulation of AMCA around the nucleus fortuitously acts as an excellent counterstain—ε-positive erythroblasts fluoresce blue and ε-negative erythroblasts autofluoresce in the SpectrumAqua™ channel, clearly identifying the location of the cells and demarcating their nuclear boundaries.

Panels B, C & D show fetal whole blood at 9 weeks' gestation. In panel B, the SpectrumAqua™ channel was used, allowing the visualization of autofluorescing heme-containing cells. One NRBC is positive for ε and the other is negative. Panel C shows the same group of cells viewed with the Red and Green filters switched on to show X and Y signals. Panel D shows the same group of cells, viewed with the SpectrumAqua™ filter off.

Panels E & F show representative ε-positive and ε-negative cells as seen in a mixing experiment of male fetal erythroblasts in never-pregnant adult female nucleated cells in a ratio of 10⁻⁵. Panel E shows a ε-positive male fetal erythroblast. Panel F shows a nearby ε-negative female nucleated cell. The autofluorescence of the cell through the green channel was deliberately potentiated to demonstrate the outline of the female leukocyte.

Panel G shows a mixing experiment of ε-positive male fetal erythroblast with K562 cells cultured in the absence of hemin. One of the X-chromosome signals in both neighbouring K562 cells are beyond the focal plane captured.

Panel H shows a mixing experiment of first trimester, ε-positive, male fetal erythroblasts with female adult bone marrow-derived erythroblasts. ε-positive fetal NRBC stained with AMCA is clearly distinguished from the AMCA-negative cell. Two X-signals are visible in the female erythroblast with demonstrable autofluorescence of heme through Red. The Y-probe is easily visualized in the male erythroblast, but the X-signal is only barely visible at the nuclear periphery in this focal plane.

The median hybridization efficiency for two FISH signals per AMCA-positive nucleated cell was 97%, comparable to 98% (n=5 sample pairs; z=0.74; NS) obtained in control slides of male and female lymphocytes.

All K562 cells cultured in 0.1 mM hemin were positive for epsilon (n=1500; 3 samples of 500 cells each) whereas no adult NRBC (n=1,000; 5 samples of 200 cells each) or white blood cell (n=250,000; 5 samples of 50,000 cells each) expressed ε-globin protein. Specificity was thus 100%. In sample mixtures (n=6 experiments), the technique was sensitive enough to consistently identify one ε-positive fetal NRBC among 10⁵ adult white blood cells (p<0.001) and 10⁶ erythrocytes, and distinguished between male fetal and adult female erythroblasts (panels E, F, G & H). This sensitivity exceeds typical MACS-based protocols (1 in 10⁴ [52-54]) and is comparable to PCR-based methods used to identify fetal DNA in enriched samples [55, 56].

Detecting ε-positive cells was simple. Viewed through the Blue filter, these cells were bright blue against a black background, rendering their identification against a contaminating maternal background amenable to automation. The high chromosomal FISH hybridization efficiency is advantageous where diagnosis is reliant upon few cells only. Conventional immunoenzymatic staining with VBS does not allow similar hybridization efficiency [8], possibly because its dense precipitate hampers penetration of FISH probes into the nucleus.

Testing Blood Sample from Pregnant Female

A sample of peripheral venous maternal blood was taken from a patient immediately after termination of pregnancy (gestation period of 11 weeks, 5 days). The sex of the fetus was determined by testing fetal blood using conventional FISH and Carnoy's fixative.

Nucleated cells in the maternal blood sample were recovered using ammonium chloride lysis, with fetal red blood cells protected by inhibiting carbonic anhydrase with acetazolamide. Maternal white blood cells were depleted using anti-CD45 antibody conjugated with MACS beads. After passing through a MACS column [57], red cells in the remaining fraction were selected using anti-glycophorin A, also by MACS. All remaining cells were spread onto a glass slide and simultaneous fluorescence immunocytochemistry and chromosomal FISH was performed as described in the previous example.

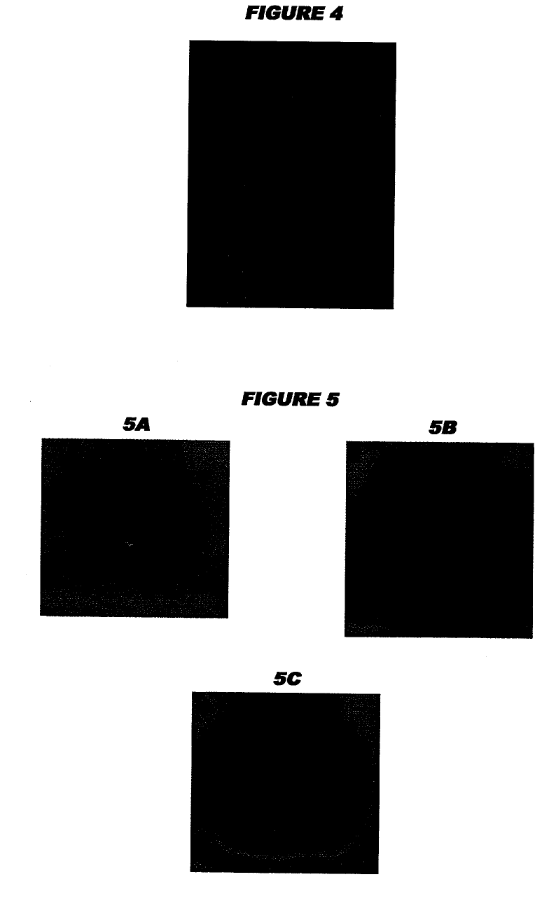

As shown in FIG. 4, ε-globin positive XY cells were visible.

In separate experiments, six maternal blood samples between weeks 8 and 11 of pregnancy were studied: three obtained immediately (≦5 minutes) post-termination and three prior to termination. Recovered fetal cells were subjected to fluorescence immunocytochemistry for

ε-globin and chromosomal FISH as described above. After this analysis had been performed, FISH was performed on fetal trophoblast tissue for confirmation of gender.

In the three post-termination samples, the number of fetal erythroblasts retrieved per 35 ml maternal blood were: 27, 9 and 14. These were all ε-globin^{+ve} (100%; p<0.001) and there were no other ε-globin^{+ve} cells (p<0.001). Of the three erythroblast samples, FISH indicated two male and one female. FISH performed on the fetuses themselves confirmed these predictions. FIG. 5 shows one male (5A) and one female (5B) erythroblast.

In the three pre-termination samples, two ε-globin^{+ve} XY fetal cells were identified (FIG. 5C). No ε-globin^{-ve} XY cells or ε-globin^{+ve} XX cells were identified.

ε-Positive Fetal Erythroblast Frequency in First Trimester Fetal Blood

Fetal whole blood was collected as described above for investigation of ε-globin expression. Nucleated cell concentrations within fetal blood were calculated using a hemocytometer and erythroblast frequency was determined by examining 200 cells per slide after Wright's staining. The relative frequencies of primitive and definitive lineage erythroblasts [58] within circulating fetal blood were determined by immunostaining all fetal NRBCs with anti-glycophorin A. This ensured that only erythroid nucleated cells were analyzed. Uniform staining of both types of fetal NRBCs by this antibody allowed study of their changing proportions across the first trimester.

As shown in FIG. 2A, the frequency of ε-positive erythroblasts in circulating fetal blood declined linearly to reach almost negligible levels by 14 weeks.

To determine the frequency of primitive and definitive lineage cells that express ε-globin, nine representative slides from samples between 9 and 12 weeks were selected and 200 cells of each lineage were examined on each slide for ε-globin staining.

The total nucleated cell concentration of fetal blood remained similar between 8-12 weeks, after which it fell sharply.

| Time | Mean total nucleated cells | Proportion of erythroblasts |
|---|---|---|
| (weeks) | (× 10⁶/ml) | (%) |
| 8-9⁺⁶ | 80.5 (95% CI, 67.3-93.7; | 96.4% (95% CI, 95.1-97.7%) |
| | n = 17) | |
| 10-11⁺⁶ | 93.3 (95% CI, 67.8-118.8; | 93.8% (95% CI, 92.7-94.9%) |
| | n = 12) | |
| 12-13⁺⁶ | 20.4 (95% CI, 14.9-26.0; | 90.6% (95% CI, 89.3-91.9%) |
| | n = 7). | |

Thus the total erythroblast concentration in fetal blood after 12 weeks gestation was markedly lower than that before 12 weeks (z=4.0; n=36; p<0.001). The frequency of primitive erythroblasts fell progressively across the first trimester to reach negligible levels by 14 weeks (FIG. 2B). This was accompanied by a reciprocal rise in the frequency of definitive NRBCs across the same gestational range such that at 12 weeks, the proportions of primitive to definitive erythroblasts were equal.

Only one definitive NRBC was ε-globin^{+ve} amongst 1,800 examined on nine representative slides between 9-12 weeks. In contrast, 100% of primitive erythroblasts expressed ε-globin. ε-globin^{+ve} anucleate erythrocytes were observed only rarely within pure first trimester fetal blood samples.

Therefore ε-globin is a suitable marker for fetal cells in first trimester blood.

It will be understood that the invention has been described by way of example only and modifications may be made whilst remaining within the scope and spirit of the invention.

### REFERENCES (the contents of which are hereby incorporated in full)

[1] Bianchi (1999) Brit. J. Haematol. 105:574-583.

[2] Stewart & Nicholson (eds) (2000) Immunophenotyping. ISBN: 0471239577.

[3] Beesley (2000) Immunocytochemistry and in Situ Hybridization in the Biomedical Sciences. ISBN: 0817640657.

[4] Beesley (ed) (1993) Immunocytochemistry: A Practical Approach. ISBN 0199632693.

[5] Javois (ed) (1999) Immunocytochemical Methods and Protocols. ISBN: 0896035700.

[6] DeMaria et al. (1996) Cytometry 25:37-45.

[7] Wang et al. (2000) Cytometry 39:224-230.

[8] Oosterwijk et al. (1998) Cytometry 32:170-177.

[9] Schwarzacher et al. (1999) Practical in situ Hybridization. ISBN: 0387915966.

[10] King et al. (2000) Mol. Diagn. 5:309-319.

[11] Pazouki et al. (1996) Acta Histochem.98:29-37.

[12] Sekizawa et al. (1998) Human Genetics 102:393-396.

[13] Price et al. (1992) Blood 80:1033-1038.

[14] Zheng et al. (1993) J Med Genet. 30:1051-1056.

[15] U.S. Pat. No. 6,169,816 (Ravkin); international patent application W098/52018.

[16] International patent application WO00/79005 (Applied Imaging Corporation)

[17] Mialhe et al. (1996) J. Histochem. Cytochem. 44:193-7.

[18] Speel et al. (1995) Histochem. J 27:833-58.

[19] Neft et al. (1997) Acta Cytologica 41:1769-73.

[20] Szollosi et al. (1995) Cytometry 20:356-361.

[21] Mosiman et al. (1997) Cytometry 30:151-156.

[22] Haugland (1999) Handbook of Fluorescent Probes and Research Chemicals (7th ed)

[23] Al-Mufti et al (2000) Human Reproduction 15:218-221.

[24] Mesker et al. (1998) Prenatal Diagnosis 18:1131-37]

[25] Pembrey et al. (1973) Lancet 1:1350-1354.

[26] Weatherall (2000) British Medical Journal 321:1117-1120.

[27] Mavrou et al. (1999) Fetal Diagn. Ther. 14:291-295.

[28] U.S. Pat. Nos. 5,731,156 and 5,962,234 (Golbus); see also WO98/18005.

[29] Choolani et al. (2001) Blood 98:554-557.

[30] Boye et al (2001) Prenat. Diagn. 21:806-812.

[31] Hogh et al. (2001) Clin. Chem. 47:645-653.

[32] Voullaire et al. (2001) Prenat. Diagn. 21:827-834.

[33] U.S. Pat. No. 5,432,054 (Saunders et al.).

[34] Bischoffet al. (1998) Am. J Obstet. Gynecol. 179:203-209.

[35] U.S. Pat. No. 6,066,459 (Garini et al.).

[36] Nolte (1998) Adv. Clin. Chem. 33:201-35.

[37] Holzgreve et al. (1999) Am. J Human Genetics 65:A177 (abstract 971).

[38] Kearney (1998) pages 161-188 of Wilkinson (ed). In situ hybridization: A practical approach. Vol The Practical Approach Series (2nd ed) (Oxford University Press)

[39] Connor et al. (1997) Essential Medical Genetics Blackwell Science, pages 86-87.

[40] Schrock et al. (1996) Science 273:494-497.

[41] Veldman et al. (1997) Nature Genetics 15:406-410.

[42] Zhen et al. (1998) Prenat. Diagn. 18:1181-85.

[43] Tanke et al. (1999) Eur. J Hum. Genet. 7:2-11.

[44] Muller et al. (1997) Human Genetics 100:271-278.

[45] Zhong et al (2001) PNAS USA 98:3940.

[46] International patent application WO00/06773.

[47] International patent application WO96/25486.

[48] Ormerod (ed) Flow Cytometry: A Practical Approach. IRL Press, ISBN 0-19 963461-0

[49] Robinson (ed) Handbook of Flow Cytometry Methods. Wiley-Liss ISBN 0-471-59634-5

[50] Campagnoli et al. (2000) Blood 95:1967-1972.

[51] Rutherford et al.(1981) Proc Natl Acad Sci USA 78:348-352.

[52] Andrews et al. (1995) Prenat. Diagn. 15:913-919.

[53] Holzgreve et al. (1992) J. Reprod. Med. 37:410-418.

[54] Wang et al. (2000) Cytometry 39:224-230.

[55] Yeoh et al. (1991) Prenat. Diagn. 11:117-123.

[56] Adinolfi (1992) Nature Genetics 1:316-318.

[57] Troeger et al. (1999) Prenat. Diagn. 19:521-6.

[58] Kelemen et al. (1979) Atlas of human hemopoietic development. NY: Springer-Verlag.

1. An antibody which can bind to an antigen present in a heme-containing cell and to which a fluorophore with an emission wavelength of between 420 nm and 500 nm is bound.

2. The antibody of claim 1, wherein said fluorophore is covalently bound to the antibody.

3. A composition comprising (a) the antibody of claim 1, and (b) a second antibody which can bind to said first antibody and to which said fluorophore is covalently bound.

4. A composition comprising (a) the antibody of claim 1, (b) a second antibody which can bind to said first antibody and to which a ligand is bound, and (c) an anti-ligand to which said fluorophore is covalently bound.

5. The composition of claim 4, wherein the ligand is biotin and the anti-ligand is selected from the group consisting of avidin and streptavidin.

6. The antibody of claim 1, wherein the cell is a human fetal erythroid cell.

7. The antibody of claim 6, wherein said antigen is the ε chain subunit of hemoglobin.

8. The antibody of claim 1, wherein the fluorophore has an emission wavelength of between 430 nm and 460 nm.

9. The antibody of claim 8, wherein the fluorophore is a 7-aminocoumarin derivative or a fluorinated 7-hydroxycoumarin.

10. The antibody of claim 9, wherein the fluorophore is AMCA or an AMCA derivative.

11. A method for identifying a cell which expresses an antigen of interest, comprising the steps of: (a) contacting a sample containing a cell or cells in a first antibody which can bind to an antigen present in a heme-containing cell and to which a fluorophore with an emission wavelength of between 420 nm and 500 nm is bound; and (b) identifying a cell or cells within the sample to which said fluorophore is bound.

12. The method of claim 11, wherein the fluorophore has an emission wavelength of between 430 nm and 460 nm.

13. The method of claim 11, wherein said fluorophore is covalently bound to said antibody.

14. The method of claim 11, which further comprises contacting the sample with a second antibody which can bind to said first antibody, and to which said flourophore is covalently bound, wherein said step (a) comprises contacting the cell in turn with (i) said first antibody and (ii) said second antibody.

15. The method of claim 11, which further comprises (a) contacting the sample with a second antibody which can bind to said first antibody and to which a ligand is bound, and an anti-ligand to which said fluorophore is covalently bound and wherein said step (a) comprises contacting the cell in turn with (i) said first antibody, (ii) said second antobody, and (iii) said anti-ligand.

16. The method of claim 15, wherein the ligand is biotin and the anti-ligand is selected from the group consisting of avidin and strepavidin.

17. The method of claim 11, wherein said cell is a human fetal erythroid cell.

18. The method of claim 17, wherein said antigen is a fetal-specific antigen.

19. The method of claim 18, wherein said antigen is the ε chain subunit of hemoglobin.

20. The method of claim 11, wherein the method is performed on a solid substrate.

21. The method of claim 20, wherein said fluorophore is detected using fluorescence microscopy.

22. The method of claim 11, wherein, prior to step (a), said sample is fixed and/or permeabilized.

23. A method for identifying a fetal cell in a blood sample taken from a pregnant female, comprising the steps of: (a) contacting the blood sample with antibody of claim 6 which can bind to a fetal-specific antigen; and (b) identifying a cell or cells in the sample to which fluorophore is bound.

24. The method of claim 23, wherein said blood sample is taken during the first trimester of pregnancy.

25. The method of claim 24, wherein said fetal-specific antigen is the ε chain subunit of hemoglobin.

26. A method for genetically testing a cell, comprising the steps of: (a) identifying a fetal cell or cells by the method of claim 23, and (b) performing genetic tests on said cell or cells.

27. The method of claim 26, wherein said genetic tests utilize FISH.

28. A method for simultaneously detecting a phenotype and a genotype of a cell, comprising the steps of: (a) contacting a cell or cells with antibody which can bind to an antigen present in a heme-containing cell and to which a fluorophore with an emission wavelength of between 420 nm and 500 nm is bound; (b) contacting said cell or cells with a nucleic acid probe, which is labeled with a second fluorophore; and (c) detecting a cell or cells in the sample to which said first and second fluorophores are bound.

29. The method of claim 28, wherein step (b) comprises contacting said cell or cells with a plurality of nucleic acid probes

30. The method of claim 29, wherein each of said plurality of probes is labeled with a different fluorophore.

31. The method of claim 28, wherein said first fluorophore gives a signal of a different color from said second fluorophore.

32. The method of claim 31, wherein the emission wavelength of the second fluorophore is more than 100 nm longer than the emission wavelength of the first fluorophore.

33. The method of claim 28, wherein the probe comprises DNA.

34. The method of claim 28, wherein said probe is a FISH probe.

35. A method for genetically testing fetal cells within a blood sample from a pregnant female, comprising the steps of: (a) contacting said blood sample with antibody which can bind to a fetal-specific antigen and to which a first fluorophore with an emission wavelength of between 420 nm and 500 nm is bound; (b) contacting said blood sample with a nucleic acid probe, wherein the probe is labeled with a second fluorophore and is specific for a genetic disorder; and (c) detecting a cell or cells in the sample to which said first and second fluorophores are bound.

36. A kit comprising (a) antibody which can bind to a fetal-specific antigen and to which a first fluorophore with an emission wavelength of between 420 nm and 500 nm is bound, and (b) a nucleic acid probe.

37. A method for permeabilizing cells, which method comprises contacting said cells with a permeabilizing agent, wherein the permeabilizing agent consists of a dilute solution of glacial acetic acid.

38. The method of claim 37, wherein the concentration of glacial acetic acid is less than 1% (v/v)

39. The method of claim 38, wherein the concentration of glacial acetic acid is around 0.25% (v/v).

40. The method of claim 39, wherein the glacial acetic acid is dissolved in methanol.

41. A kit comprising (a) a first antibody which can bind to an antigen present in a heme-containing cell; (b) a second antibody which can bind to the first antibody and to which a fluorophore with an emission wavelength of between 420 nm and 500 nm is covalently bound.

42. The kit of claim 41, wherein the cell is a human fetal erythroid cell.

43. The kit of claim 41, wherein the fluorophore is a 7-aminocoumarin derivative or a fluorinated 7-hydroxycoumarin.

44. The kit of claim 43, wherein the fluorophore is AMCA or an AMCA derivative.

45. A kit comprising (a) a first antibody which can bind to an antigen present in a heme-containing cell; (b) a second antibody which can bind to the first antibody and to which a ligand is bound; and (c) an anti-ligand to which a fluorophore with an emission wavelength of between 420 nm and 500 nm is covalently bound.

46. The kit of claim 45, wherein the cell is a human fetal erythroid cell.

47. The kit of claim 45, wherein the fluorophore is a 7-aminocoumarin derivative or a fluorinated 7-hydroxycoumarin.

48. The kit of claim 47, wherein the fluorophore is AMCA or an AMCA derivative.

49. A flow cytometry method, which method comprises labeling cells with antibody of claim 1.

50. The method of claim 49, which is a fluorescence activated cell sorting (FACS) method.
